(19) United States
(12) Patent Application Publication
(10) Pub. No.: US 2014/0154303 A1
(43) Pub. Date: Jun. 5, 2014

(54) TREATING CANCER BY INHIBITING EXPRESSION OF OLFM4, SP5, TOB1, ARID1A, FBN1 OR HAT1

(71) Applicants: Patrick Tan, Singapore (SG); Hue Kian Oh, Singapore (SG)

(72) Inventors: Patrick Tan, Singapore (SG); Hue Kian Oh, Singapore (SG)

(73) Assignees: Singapore Health Services Pte Ltd., Singapore (SG); National University of Singapore, Singapore (SG)

(21) Appl. No.: 13/984,977

(22) PCT Filed: Feb. 13, 2012
(86) PCT No.: PCT/SG2012/000040

§ 371 Date: Oct. 21, 2013

(30) Foreign Application Priority Data
Feb. 11, 2011 (SG) 201101013-9

(51) Int. Cl. A61K 31/713; C12Q 1/68; A61K 31/712; A61K 31/7105; A61K 31/7088; A61K 31/7125

(52) U.S. Cl. CPC A61K 31/713; A61K 31/7088; A61K 31/7125; A61K 31/712; A61K 31/7105; C12Q 1/6886
USPC 424/450; 514/44 A; 424/490; 424/130.1; 514/44 R; 435/6.11

(57) ABSTRACT

The present invention in one aspect relates to a method for treating cancer or enhancing a cancer treatment, the method comprising inhibiting the expression of an oligonucleotide encoding for a protein selected from the group consisting of OLFM4, SP5, TOB1, ARID1A, FBN1 and HAT1, by administering to a mammal in need of cancer treatment an effective amount of at least one silencing oligonucleotide comprising a binding motif for binding to the oligonucleotide encoding for a protein.

### TECHNICAL FIELD

The present invention relates to a therapeutic in the treatment and screening of cancer or conditions associated with cancer.

### BACKGROUND

Cancer claims millions of lives around the globe each year. Amongst a hundred or more different types of cancer occurring, lung, gastric, liver, colon and breast cancer are the most common causes of cancer death. It has been observed that gastric cancer, being the second leading cause of global cancer mortality, has a particularly high incidence rate in Asian and South American countries. Most gastric cancer patients are diagnosed with advanced stage disease and show extremely poor prognosis. The five-year survival rate for patients with stage II disease ranges from 30-50%, falling to 10-25% for state III patients. Disease recurrence is also common in gastric cancer, as with the case of breast cancer, for example.

Cancer generally involves unregulated cell growth and is a difficult class of disease to treat. Conventional cancer treatments such as conventional radiation therapy or chemotherapy have not always been successful. In fact, drug resistance is a common problem encountered in chemotherapy. Also, the conventional cancer therapies do not specifically target and treat the affected cells and are known to have severe side effects.

Genetic factors such as mutations in classical tumor suppressor genes and oncogenes, and gene amplifications, deletions or inactivation have been implicated in cancer development.

Small RNA molecules have also been found to play an important role in human cancer. A small RNA molecule may exert either an oncogenic or tumor suppressive effect.

However, small RNA or other oligonucleotide-based therapeutic approaches or gene therapies are not yet common or available as new treatment strategies for cancer.

There is a need to provide a therapeutic or alternative treatment that overcomes, or at least ameliorates, one or more of the disadvantages associated with a conventional cancer treatment as described above.

### SUMMARY

According to a first aspect, there is provided a method for treating cancer or enhancing a cancer treatment, the method comprising inhibiting the expression of an oligonucleotide encoding for a protein selected from the group consisting of OLFM4, SP5, TOB1, ARID1A, FBN1 and HAT1, by administering to a mammal in need of cancer treatment an effective amount of at least one silencing oligonucleotide comprising a binding motif with a core nucleotide sequence of UCCUGUAC, or at least one silencing oligonucleotide targeting the 3′ untranslated region of the oligonucleotide encoding for a protein selected from the group consisting of OLFM4, SP5, TOB1, ARID1A, FBN1 and HAT1.

According to a second aspect, there is provided a method of inducing apoptosis in a mammal in need thereof, the method comprising administering to the mammal an effective amount of at least one silencing oligonucleotide comprising a binding motif with a core nucleotide sequence of UCCUGUAC to bind and thereby inhibit an anti-apoptotic protein in the mammal.

According to a third aspect, there is provided a method of screening for cancer, the method comprising screening for a silencing oligonucleotide comprising a binding motif with a core nucleotide sequence of UCCUGUAC, or an oligonucleotide encoding for a protein and including a matching nucleotide sequence being complementary to the nucleotide sequence of UCCUGUAC.

### DEFINITIONS

The following are some definitions that may be helpful in understanding the description of the present invention. These are intended as general definitions and should in no way limit the scope of the present invention to the defined terms alone. The definitions are put forth for a better understanding of the following description.

The term “small RNA molecules” or grammatical variants thereof as used herein refers to RNA molecules which may be double stranded or single-stranded and may comprise from about 3 to about 200 bases or base pairs in length. For example, a small RNA molecule as defined herein may comprise from about 4 to about 180 bases or base pairs, from about 5 to about 150 bases or base pairs, from about 6 to about 120, from about 7 to about 90, from about 8 to about 80 bases or base pairs, from about 10 to about 60 base or base pairs, from about 20 to about 40 bases or base pairs, or from about 20 to about 30 bases or base pairs in length. The small RNA molecules may come in many forms, for example, small interfering (si) RNAs, single helix (sh) RNAs, small temporal (st) RNAs, tiny noncoding RNAs or micro (mi) RNAs. The small RNA molecules may be derived from endogenic or exogenic sources. They can control mRNA stability or translation, inhibit the expression of an oligonucleotide encoding for a protein or target epigenetic modifications to specific regions of the genomes in eukaryotes.

The term “oligonucleotide” as used herein refers to a nucleotide sequence encoding for a protein or a fragment thereof.

The term “silencing oligonucleotide” as used herein refers to an oligonucleotide comprising a particular nucleotide sequence that may target and bind to an oligonucleotide encoding for a protein, thereby ‘switching-off’ or inhibiting the activity of the oligonucleotide encoding for the protein, and thereby preventing the translation of the protein. A silencing nucleotide as defined may comprise about 3 to about 200 nucleotides. For example, the silencing oligonucleotide may comprise from about 4 to about 180 nucleotides, from about 5 to about 150 nucleotides, from about 6 to about 120 nucleotides, from about 7 to about 90 nucleotides, from about 8 to about 80 nucleotides, from about 10 to about 60 nucleotdies, from about 20 to about 40, or from about 20 to about 30 nucleotides. A silencing oligonucleotide as defined herein may be a small RNA molecule including, but not limited to, a small interfering RNA, single helix RNA, small temporal RNA, tiny noncoding RNA or micro RNA.

The term “small interfering RNA” (si-RNA), which may also be known as “short interfering RNA” or “silencing RNA”, refers to a particular class of ‘small RNA molecules’ as defined above. The term “small interfering RNA” or “si-RNA” as used herein includes, but is not limited to, a double-strand RNA (dsRNA), with each strand having a 5′ phosphate group and a 3′ hydroxyl group. This structure is the result of processing by dicer, an enzyme that converts either long dsRNAs or small hairpin RNAs into siRNAs. Once processed by the dicer, the si-RNA may then be incorporated in the si-RNA induced silencing complex (RISC) to facilitate the cleavage ad degradation of its recognized mRNA. The si-RNAs play a notable role in the RNA interference (RNAi) pathway, where they interfere with the expression of a specific oligonucleotide encoding for a protein.

The term “micro RNA”, “mi-RNA” or “miR” as used herein refers to a particular class of “small RNA molecules” as defined above. In particular, the term “micro RNA”, “mi-RNA” or “miR” may refer to a non-coding RNA comprising from about 3 to about 200, from about 4 to about 180, from about 5 to about 150, from about 6 to about 120, from about 7 to about 90, from about 8 to about 80, from about 10 to about 60, from about 20 to about 40 or from about 20 to about 30 nucleotides in length, which hybridizes to and regulates the expression of a coding RNA. The mi-RNAs as referred to may be single or double-stranded and may be obtained from a micro RNA precursor, such as a hairpin RNA precursor, by natural processing routes (e.g. using intact cells or cell lysates) or by synthetic routes (e.g. using isolated processing enzymes, such as the dicer enzyme or RNAase III). Alternatively, the mi-RNAs may be obtained directly by biological or chemical synthesis without the involvement of a precursor. Similar to the si-RNAs defined above, the mi-RNAs can silence the activity of an oligonucleotide encoding for a protein by blocking its translation in plants and animals. The term “mi-RNA”, “micro-RNA” or “miR” as used herein generally refers to a maturemi-RNA or mi-RNA sequence, not a precursor thereof.

The term “precursor” as used herein may refer to a non-coding RNA having a hairpin structure which contains a mi-RNA or mi-RNA sequence, which may be cleaved off the precursor structure. The “precursor” in certain embodiments may be the product of cleavage of a primary mi-RNA transcript; however, a precursor mi-RNA may also be produced directly by biological or chemical synthesis from an endogenous or exogenous source.

The term “cell proliferative disorder” or “cell proliferation” or “proliferation” as used herein refers to an unwanted or uncontrolled cellular proliferation of excessive or abnormal cells, such as in neoplastic or hyperplastic growth, whether in vitro, in vivo or ex vivo. Exemplary proliferative conditions include, but are not limited to, pre-malignant and malignant cellular proliferation, such as malignant neoplasms, tumours (e.g. solid tumours), cancers, and the like.

The term “apoptosis” as used herein refers to a process of programmed cell death, in which the cell undergoes a series of changes upon detection of an intrinsic signal (resulting from cellular stress) or extrinsic signal (resulting from binding of a death inducing ligand to cell surface) instructing the cell to undergo apoptosis. In one embodiment, the cell may begin to shrink following the cleavage of actin filaments, for example, in the cytoskeleton. Subsequently, chromatin may break down in the nucleus and lead to nuclear condensation resulting in the nuclei of an apoptotic cell taking on a ‘horse-shoe’ like appearance. Finally in this embodiment, the cells may continue to shrink and package themselves into a form that enables their removal by macrophages. Thus, in apoptosis, cells play an active role in their own death.

The term “anti-apoptotic” as used herein refers to a protein or an oligonucleotide (which may be an oligonucleotide encoding for a protein or a silencing nucleotide) which acts to prevent apoptosis of a cell, in particular a cell experiencing stress, a cell received signal to undergo apoptosis or a cell undergoing abnormal cell proliferation.

The term “administering” and variations of this term including “administer” and “administration”, includes contacting, applying, delivering or providing a compound or molecule or formulation of the invention on a surface, or to an organism or to a specimen of the organism, by any appropriate means. In one embodiment, the specimen may be a cell or tissue sample of the organism.

The term “modulating” as used herein generally means regulating a biological process or activity, such as the expression of an oligonucleotide encoding for a protein or the expression of a silencing oligonucleotide, inside a cell or inside a mammal. The term “modulating” encompasses the situations of up-regulating or down-regulating with respect to the process or activity. For example, ‘modulating’ the expression of an oligonucleotide encoding for a protein may be to increase or decrease the level of expression with respect to the oligonucleotide encoding for the protein.

The term “systemic administration” as used herein refers to a route of administering a compound, molecule or formulation of the invention, such as a silencing oligonucleotide, by enteral administration through the gastrointestinal track, or parenteral administration through the skin or a mucous membrane, for example.

The term “effective amount” as used herein refers to an amount sufficient to induce a desired effect in a subject, such as inhibition of a target oligonucleotide encoding for a protein in a cell or a mammal, without the amount being toxic to the subject. The exact amount required will vary from subject to subject depending on factors such as the species being treated, the age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art using only routine experimentation.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of a method for treating cancer or enhancing a cancer treatment will now be disclosed.

The method may comprise inhibiting the expression of an oligonucleotide encoding for a protein selected from the group consisting of OLFM4, SP5, TOB1, ARID1A, FBN1 and HAT1, by administering to a mammal in need of cancer treatment an effective amount of at least one silencing oligonucleotide comprising a binding motif with a core nucleotide sequence of UCCUGUAC, or at least one silencing oligonucleotide targeting the 3′ untranslated region of the oligonucleotide encoding for a protein selected from the group consisting of OLFM4, SP5, TOB1, ARID1A, FBN1 and HAT1.

In one embodiment, the oligonucleotide comprises a nucleotide sequence encoding for a protein or fragment thereof.

In one embodiment, the silencing oligonucleotide comprises from about 3 to about 200 nucleotides. For example, the silencing oligonucleotide may comprise from about 4 to about 180 nucleotides, from about 6 to about 120 nucleotides, from about 8 to about 80 nucleotides, from about 8 to about 70, from about 10 to about 60 nucleotides, from about 20 to about 40, from about 20 to about 30 nucleotides, or from about 10 to about 20 nucleotides.

In one embodiment, one, two or more silencing oligonucleotides may be administered.

In one embodiment, the silencing oligonucleotide may be derived from an existing or natural sequence.

In one embodiment, the silencing oligonucleotide may be generated in any manner, for example by chemical synthesis, DNA replication, reverse transcription or any combination thereof.

In one embodiment, the silencing oligonucleotide may be a RNA or DNA fragment or sequence, and may be single-stranded or double-stranded.

In one embodiment, the silencing oligonucleotide may be in the form of a si-RNA, sh-RNA or mi-RNA.

In one embodiment, the silencing oligonucleotide may be a si-RNA in the form of a double-strand RNA (dsRNA), with each strand having a 5′ phosphate group and a 3′ hydroxyl group.

In one embodiment, the silencing oligonucleotide may be a micro RNA (mi-RNA) or a precursor thereof.

In one embodiment, the silencing oligonucleotide may be a mi-RNA in the form of a non-coding RNA comprising from about 5 to about 70, from about 15 to about 50 or from about 20 to about 30 nucleobases in length.

In one embodiment, the silencing nucleotide is selected from the group consisting of hsa-miR-623, hsa-miR-134, hsa-miR-181c, hsa-miR-654-5p, hsa-miR-936, hsa-miR-939, kshv-miR-K12-3, hsa-miR-550, hsa-miR-486-5p, hsa-miR-575, hcmv-miR-UL70-3p, Hsa-miR-638, hsvl-miR-H1, hsa-miR-139-3p, hsa-miR-202, hsa-miR-378, hsvl-miR-LAT, hsa-miR-596, hsa-miR-188-5p, hsa-miR-28-3p, hsa-miR-30d, hsa-miR-572, hsa-miR-1225-5p, hsa-miR-345, hsa-miR-30a, hsa-miR-671-5p, hivl-miR-H1, hsa-miR-148a, hsa-miR-222, hsa-hsa-miR-564, hsa-miR-193b, hsa-miR-125a-3p, hsa-miR-370, hsa-miR-375, hsa-miR923, hsa-miR-513c, hsa-miR-513a-5p, hsa-miR-494 and hsa-miR-513b, hsa-miR-486-5p, hsa-let-7d*, hsa-miR-328, hsa-miR-32*, hsa-miR-1227, hsa-miR-206. hsa-miR-1229, hsa-miR-595 and hsa-miR-631, or a precursor of these mi-RNAs.

In another embodiment, the mi-RNA is selected from the group consisting of hsa-miR-375, hsa-miR-148a, miR-671-5p, hsa-miR-30a, hsa-miR-1225-5p, hsa-miR-572, hsa-miR-188-5p, miR-139-3p, hsa-miR-638, hsa-miR-486-5p, hsa-miR-550, miR-939, hsa-miR-936, hsa-miR-181c, miR-134, hsa-miR-623, hsa-let-7d*, hsa-miR-328, hsa-miR-32*, hsa-miR-1227, hsa-miR-206. hsa-miR-1229, hsa-miR-595 and hsa-miR-631, or a precursor of these mi-RNAs.

In yet another embodiment, the mi-RNA is hsa-miR-486-5p (referred to as miR-486 hereafter) of SEQ ID NO:1, or a precursor thereof of SEQ ID NO:2.

In one embodiment, the silencing oligonucleotide is a precursor of a mi-RNA or si-RNA.

In one embodiment, the precursor may be a hairpin RNA precursor containing a mi-RNA or mi-RNA sequence.

In one embodiment, the precursor may be a hairpin RNA precursor containing miR-486.

In one embodiment, the oligonucleotide encoding for a protein is selected from the group consisting of OLFM4, SP5, TOB1, ARID1A, FBN1 and HAT1, or the group consisting of OLFM4, SP5, TOB1 and ARID1A or the group consisting of OLFM4 and SP5.

In one embodiment, each of the oligonucleotide encoding for a protein OLFM4, SP5, TOB1, ARID1A, FBN1 and HAT1 may serve a regulatory role in cancer. For example, oligonucleotides encoding for TOB1 and ARID1A may be associated with tumor suppressive functions.

In one embodiment, the oligonucleotides encoding for a protein may be selected from the group consisting of OLFM4, SP5, TOB1 and ARID1A.

In one embodiment, the oligonucleotide encoding for a protein may be selected from the group consisting of OLFM4 and SP5.

In one embodiment, the oligonucleotide encoding for a protein is OLFM4 (Olfactomedin-4), which is an anti-apoptotic protein.

In one embodiment, the oligonucleotide encoding for OLFM4 has at least one binding site for binding with the binding motif with a core nucleotide sequence of UCCUGUAC of the silencing oligonucleotide.

In one embodiment, the at least one binding site is located in the 3′-UTR region of the oligonucleotide encoding for the OLFM4 protein.

In one embodiment, a binding site of the oligonucleotide encoding for the OLFM4 protein comprises a nucleotide sequence of SEQ ID NO:3.

In one embodiment, the oligonucleotide encoding for OLFM4 having the binding site comprising a nucleotide sequence of SEQ ID NO:3 is capable of binding with a silencing oligonucleotide in the form of a mi-RNA, for example. The exemplary mi-RNA has the binding motif with a core nucleotide sequence of UCCUGUAC. In this embodiment, SEQ ID NO:3 and the core nucleotide sequence of the binding motif are complementary to one another.

In one embodiment, a mi-RNA in the form of miR-486 or a precursor thereof is bound to the oligonucleotide encoding for the OLFM4 protein. The binding of miR-486 or a precursor thereof to the oligonucleotide encoding for the OLFM4 protein prevents translation of and thereby inhibits the expression of the anti-apoptotic OLFM4 protein.

In one embodiment, inhibition of expression of the anti-apoptotic OLFM4 protein promotes apoptosis of cells with abnormal proliferation in the mammal.

In one embodiment, the silencing oligonucleotide is downregulated in the mammal.

In one embodiment, a mi-RNA in the form of miR-486 is downregulated in the mammal. The downregulation of miR-486 may result in the upregulation of the oligonucleotide encoding for an anti-apoptotic protein, such as the OLFM4 protein.

In one embodiment, the cancer to be treated by the disclosed method includes, but is not limited to, gastric cancer, colon cancer, breast cancer and lung cancer. Other cancer types, for example a blood cancer such as leukemia, may also be treated by the disclosed method.

In one embodiment, the cancer to be treated is gastric cancer.

In one embodiment, the cancer to be treated is intestinal-type gastric cancer or diffuse-type gastric cancer.

In one embodiment, the silencing oligonucleotide is in an isolated form.

In one embodiment, the isolated silencing oligonucleotide comprises a chemical modification of one or more nucleotides. The modification may take place at the guanine, uracil or adenosine base, for example.

In one embodiment, the chemical modification comprises a phosphate backbone modification, a modified sugar moiety, a modified nucleotide, or a modified terminal.

In one embodiment, the phosphate backbone modification is selected from the group consisting of phosphorothioate modification, methylphosphonate modification, phosphotriester modification, phosphordithionate modification and phosphoselenate modification.

In one embodiment, the silencing oligonucleotide is provided in a formulation, which may be administered in an effective amount to a mammal or a mammalian cell for altering, e.g. decreasing, the level of an apoptotic protein in the cell or the mammal.

In one embodiment, the silencing oligonucleotide is provided in the form of an emulsion containing the silencing oligonucleotide.

In one embodiment, the silencing oligonucleotide is formulated with a delivery vehicle.

In one embodiment, the delivery vehicle is a nanoparticle, in the form of liposome, or a peptide, or an aptamer, or an antibody, or a polyconjugate or microencapsulation.

In one embodiment, the lipsome is a stable nucleic acid-lipid particle (SNALP), or dioleoyl, phosphatidylcholine (DOPC)-based delivery system or a lipoplex.

In one embodiment, the silencing oligonucleotide may be administered by intravenous, intramuscular, transcutaneous, subcutaneous, intranasal, peroral or systemic administration. In one embodiment, the silencing oligonucleotide is formulated for systemic administration.

In one embodiment, the silencing olignucleotide is encoded with an expression vector for expression in a mammalian cell.

In one embodiment, the expression vector is a viral vector selected from the group consisting of a retroviral, adenviral, lentiviral and adeno-associated viral vector.

In one embodiment, the mammalian cell is a tumor cell.

In one embodiment, the mammal is a human, thus the mammalian cell is a human cell and the tumor cell is a human tumor cell.

There is further provided a method of inducing apoptosis in a mammal in need thereof, the method comprising administering to the mammal an effective amount of a silencing oligonucleotide comprising a binding motif with a core nucleotide sequence of UCCUGUAC to bind and thereby inhibit an anti-apoptotic protein in the mammal.

In one embodiment, the anti-apoptotic protein is OLFM4.

In one embodiment, the mammal in need thereof is a mammal with a cell proliferative disorder, such as cancer.

In one embodiment, the cancer includes, but is not limited to, gastric cancer, colon cancer, breast cancer and lung cancer.

There is further provided a method of screening for cancer, the method comprising screening for a silencing oligonucleotide comprising a binding motif with a core nucleotide sequence of UCCUGUAC, or an oligonucleotide encoding for a protein and including a matching nucleotide sequence being complementary to the nucleotide sequence of UCCUGUAC.

In one embodiment; the method of screening for cancer comprises mi-RNA profiling or deep sequencing.

In one embodiment, the method comprises screening for a silencing oligonucleotide comprising a binding motif with a core nucleotide sequence of UCCUGUAC that is down regulated in cancer. In this embodiment, a significantly decreased level of expression or even non-expression is observed with the silencing oligonucleotide, as compared to a situation of without cancer.

In one embodiment, the silencing oligonucleotide is a mi-RNA in the form of miR-486.

In one embodiment, the method comprises screening for an upregulated oligonucleotide encoding for a protein and including a matching nucleotide sequence being complementary to the nucleotide sequence of UCCUGUAC. In this embodiment, overexpression or a significantly increased level of expression is observed with the oligonucleotide encoding for a protein, as compared to a situation of without cancer.

In one embodiment, the oligonucleotide encoding for a protein is OLFM4.

In one embodiment, the method comprises obtaining blood and serum samples from a plurality of mammals; analyzing and screening the samples for an abnormally low level of miR-486 or an abnormally high expression level of OLFM4, to thereby identify mammals that might have cancer.

In another embodiment, suspicious pre-malignant lesions may be checked for their miR-486/OLFM4 levels, to assess the risk of these lesions progressing to malignant cancer.

In one embodiment, the cancer is gastric cancer.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows global identification of differentially expressed microRNAs between gastric normal tissues and tumors.

FIG. 2 shows expression of candidate tumor suppressor hsa-miR-486-5p (miR-486) in gastric tumors and cell lines.

FIG. 3 shows that miR-486 expression modulates multiple pro-oncogenic traits.

FIG. 4 shows genomic loss of miR-486 in primary gastric cancers.

FIG. 5 shows direct regulation of Olfactomedin-4 (OLMF4) by miR-486.

FIG. 6 shows downregulation of OLFM4 protein by miR-486 induces protein upregulation of GRIM-19.

FIG. 7 shows growth inhibition effects of miR-486 being counteracted by OLFM4 overexpression.

FIG. 8 shows miR-486 expression in normal gastric tissue and in matching tissue with tumour.

### DETAILED DESCRIPTION OF DRAWINGS

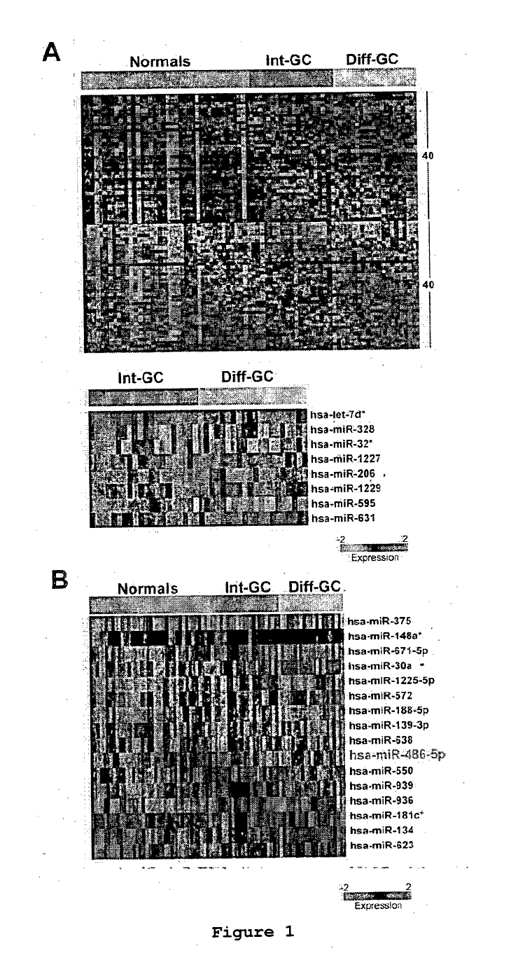

FIG. 1 shows global identification of differentially expressed mi-RNAs between gastric normal tissue and gastric tumor tissues. FIG. 1 comprises Figures A and B. The top panel of FIG. 1A is an expression heatmap showing expression of 80 differentially expressed mi-RNAs, including 40 normal gastric tissues (“Normals”) and 40 gastric cancer (GC) tissues (“Int-GC” and “Diff-GC”) (FDR<0.01, using Significance Analysis of Microarrays). The bottom panel of FIG. 1A shows differential expression of mi-RNAs between intestinal- and diffuse-type gastric cancers analyzed from a separate SAM run (FDR<0.01). FIG. 1B is a heatmap showing differential expression of 16 candidate tumor suppressor mi-RNAs. Expression of miR-486 (has-miR-486-5p) is highlighted in big font.

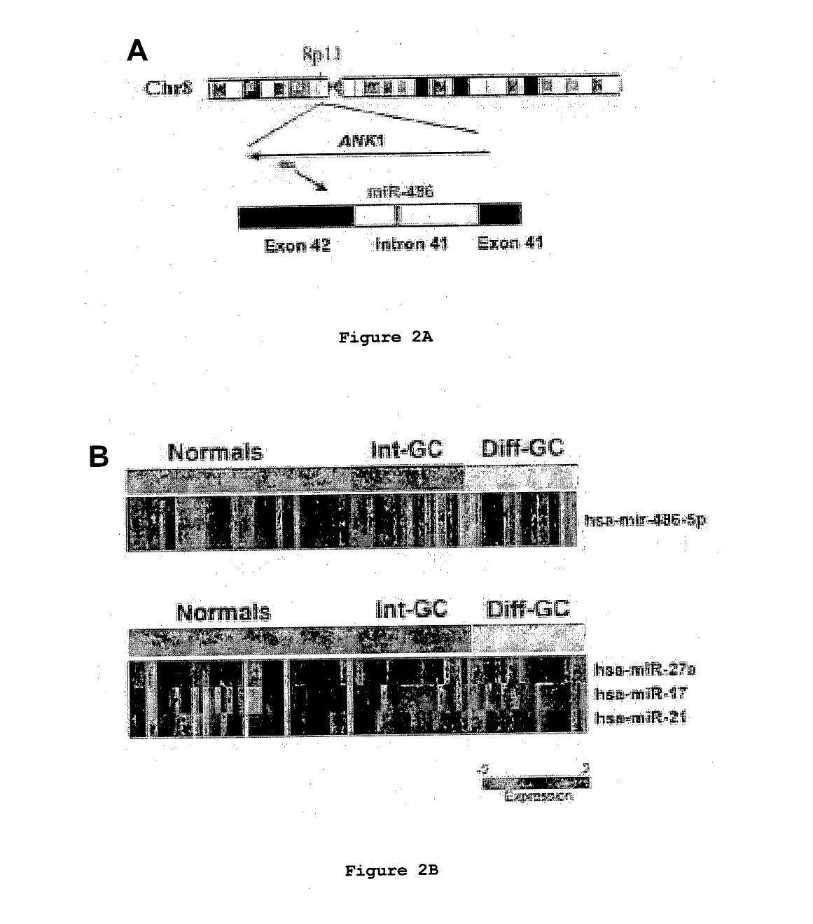

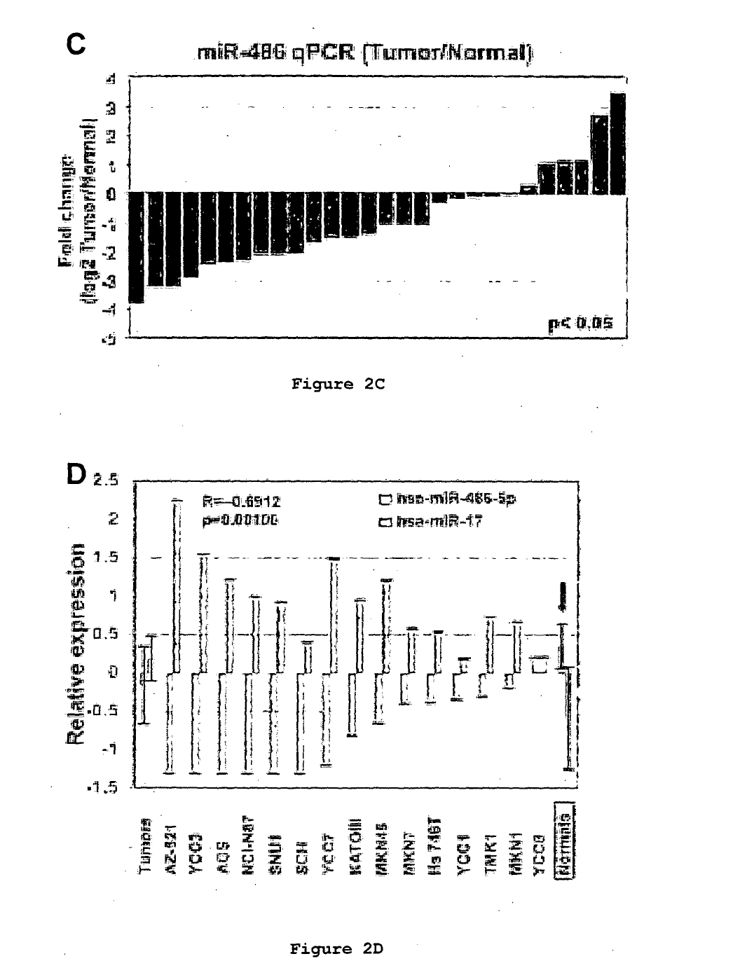

FIG. 2 shows the expression of a particular candidate tumor suppressor miR-486 in gastric tumors and cell lines. FIG. 2 comprises FIGS. 2A-2D. FIG. 2A shows the genomic location of miR-486. miR-486 is located on chromosome 8p11 within the ANK1 gene, between exons 41 and 42. miR-486 is transcribed from the same strand from an alternative promoter located in intron 40 of the ANK1 gene. FIG. 2B shows the reciprocal expression of miR-486 compared to three previously reported oncogenic mi-RNAs (miR-17, miR-21, and miR-27a) across gastric normal tissues and gastric cancers. FIG. 2c is the result of a Quantitative PCR (qPCR) analysis and shows the relative expression of miR-486 in 29 primary gastric cancer tissues compared to matched adjacent normal tissues. Quantifications were measured using TaqMan real-time PCR. Each column represents an individual tumor/normal pair. Fold-changes (tumor/normal) were transformed to log 2 values (y-axis). P-values denotes the significance of fold changed observed. FIG. 2D shows the expression of miR-486 and miR-17 in primary gastric tissues (normal and tumors) and in gastric cancer cell lines. The x-axis depicts primary gastric cancers (first column, n=40, median), 15 gastric cancer cell lines, and primary normal gastric tissues (last column, n=40, median). Expression of miR-486 is shown in the y-range of 0 to 2.5 while miR-17 is shown in the y-range of 0 to −1.5. Error bars indicate respective standard deviations across tumors or normals. R denotes the Pearson correlation between the two mi-RNAs. P-values denoting the significance of the correlation coefficient R. Cell lines selected for functional analysis include YCC3, AGS, SCH and YCC6.

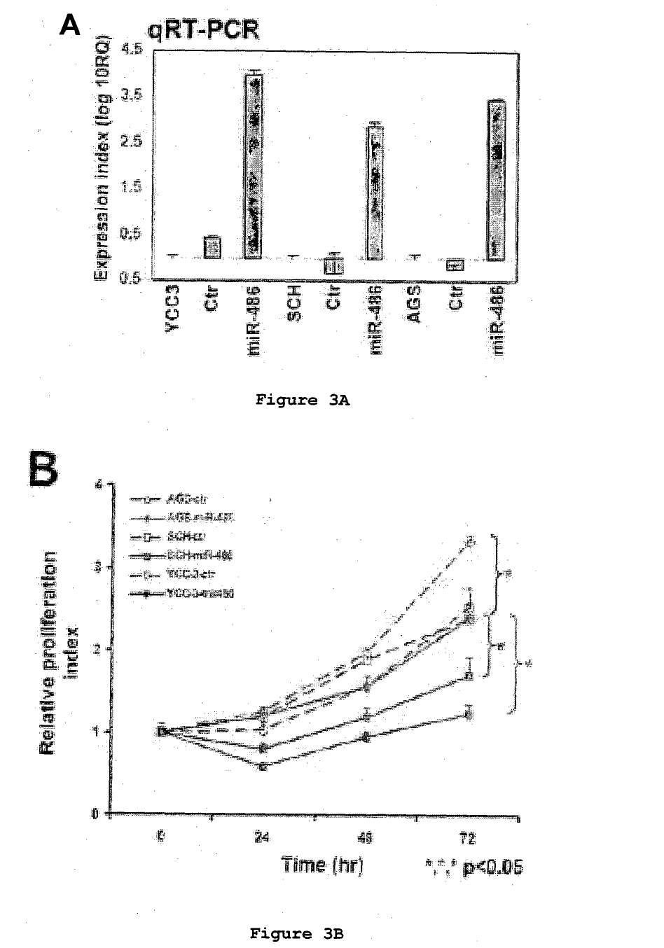

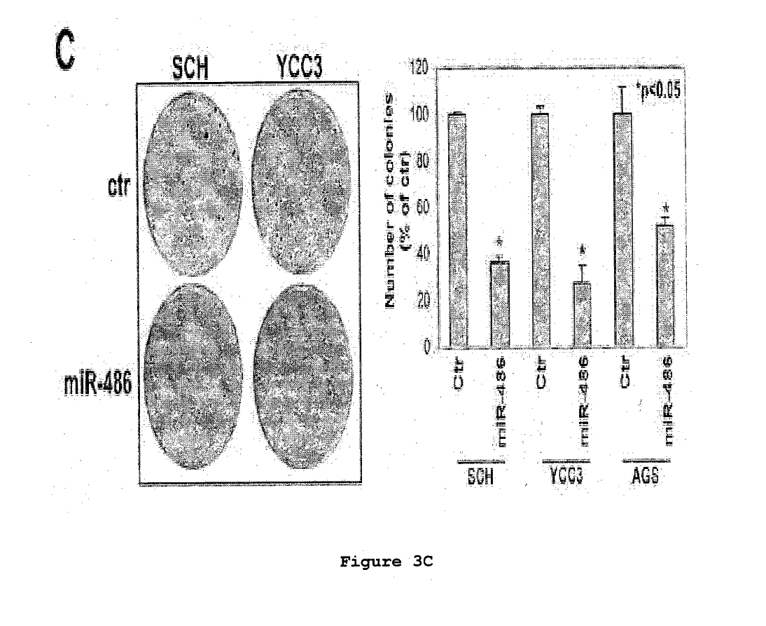

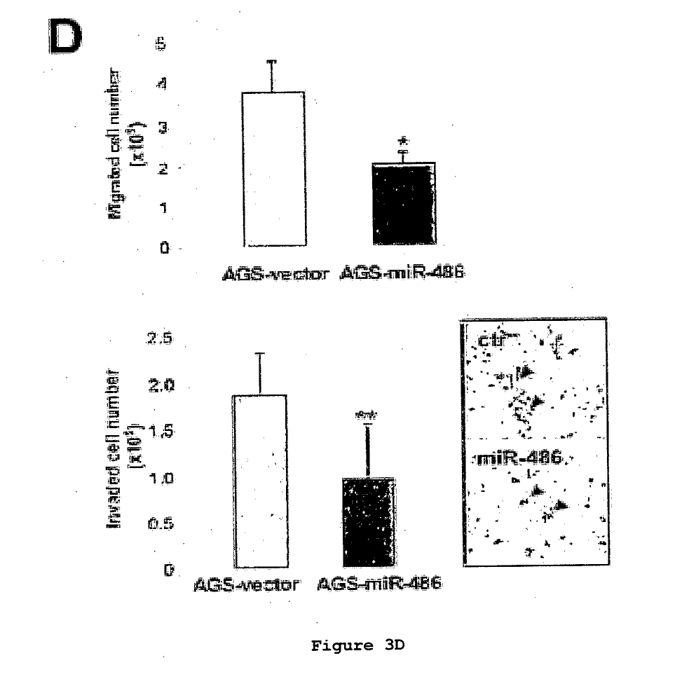

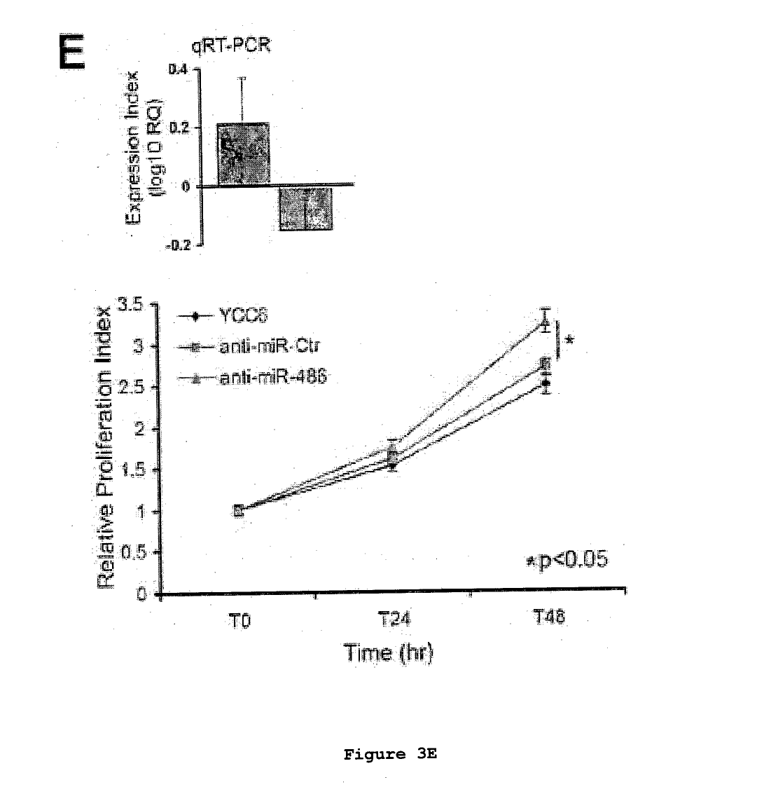

FIG. 3 shows that miR-486 expression modulates multiple pro-oncogenic traits. FIG. 3 comprises FIGS. 3A-3E. FIG. 3A shows the restoration of miR-486 expression in gastric cancer cell lines. YCC3, SCH and AGS cells were transfected with control of miR-486 precursors, qRT-PCR was performed to assess relative miR-486 expression. FIG. 3B shows that miR-486 expression suppresses cellular proliferation in gastric cancer cell lines. miR-486 transfected cell lines were assessed for cell proliferation at 24, 48 and 72 hr post-transfection. Triplicate experiments were performed for each set (*, p<0.05, t-test; points represent means; bars represent standard deviations). FIG. 3c shows that miR-486 expression suppresses anchorage-independent cell growth. The left panel shows a soft-agar colony formation assay reduction demonstrating reduced in colony numbers in SCH and YCC3 stably transfected with miR-486 or empty vector control. The right panel shows the quantification of colonies observed. Triplicate experiments were performed for each cell lines (columns represent means; bars represent standard deviations; *, p<0.05, t-test). FIG. 3D shows that miR-486 reduces motility and invasiveness of AGS cells. The top-panel shows the migration of AGS cells stably expressing miR-486 or vector controls, as measured using a Transwell migration assay (columns represent mean; bars represent standard deviations; *, p<0.05). The bottom panel of FIG. 3D shows the invasion of AGS cells stably expressing miR-486 or vector controls, as measured using a Matrigel assay. The insert of FIG. 3D shows phase contrast microscopy of the stained cells in Matrigel (columns represent means; bars represent standard deviations; **, p=0.059). FIG. 3E shows that the inhibition of miR-486 expression promotes cellular proliferation in YCC6. In the top panel, miR-486 inhibitor and negative control inhibitor (anti-miR-486 (column below y=0 line) and anti-miR-ctr (column above y=0 line)) transfected cells were assessed for miR-486 expression using qRT-PCR. In the bottom panel, cell proliferation levels were assayed at 24 and 48 hr post-transfection. Triplicate experiments were performed for each set (points represent means; bars represent standard deviations; significances for the difference in growth denoted by *, p<0.05).

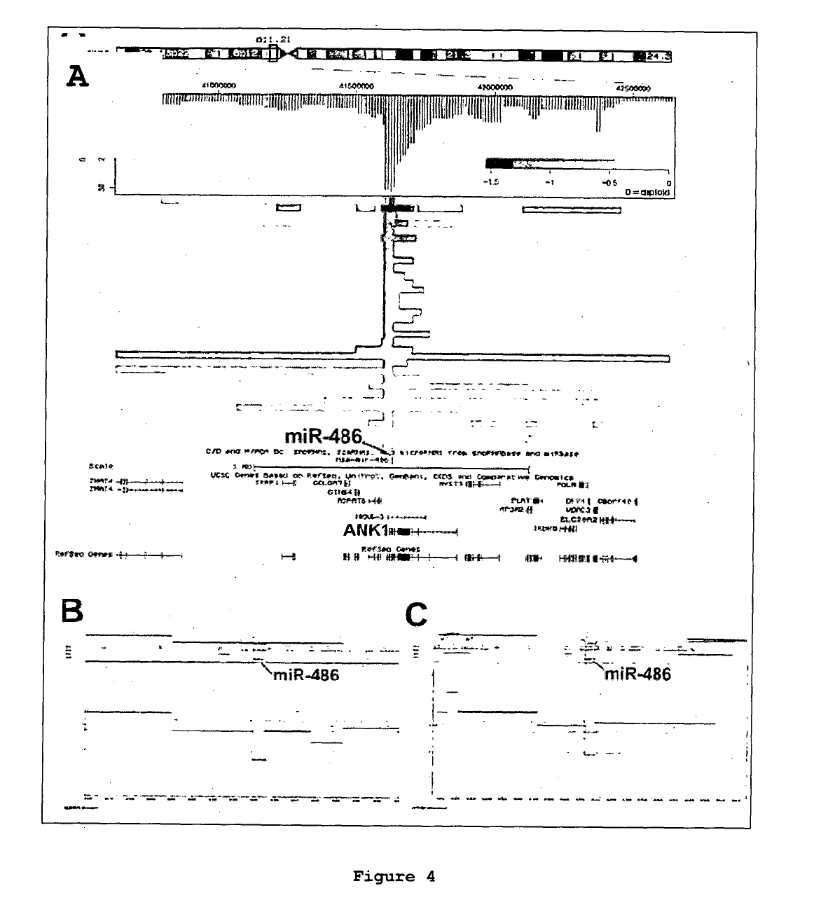

FIG. 4 shows the genomic loss of miR-486 in primary gastric cancers. FIG. 4 comprises FIGS. 4A and 4B. FIG. 4A shows the recurrent genomic loss of miR-486. The vertical bars highlight the miR-486 locus. The top panel shows the genomic location of miR-486 on Chr 8p11.21. The middle panel is a histogram showing frequency of genomic loss in this region across 106 primary gastric cancers. 28% of the gastric cancers (i.e. 30 tumors) are observed to exhibit loss of the miR-486 locus. The lower panel shows genomic loss of the miR-486 locus in individual samples, only samples with miR-486 are shown. The shade or color gradient depicts the extent of copy number deletion. The bottom panel shows genome browser view of the Chr 8p11.21 region showing miR-486 and adjacent genes such as ANK1. FIGS. 4B and 4C show focal deletion of miR-486 in two gastric tumor samples: 2000088 and 990187. The copy number log-ratio data is shown together with segments identified. The miR-486 locus (with focal deletion) is highlighted or shaded.

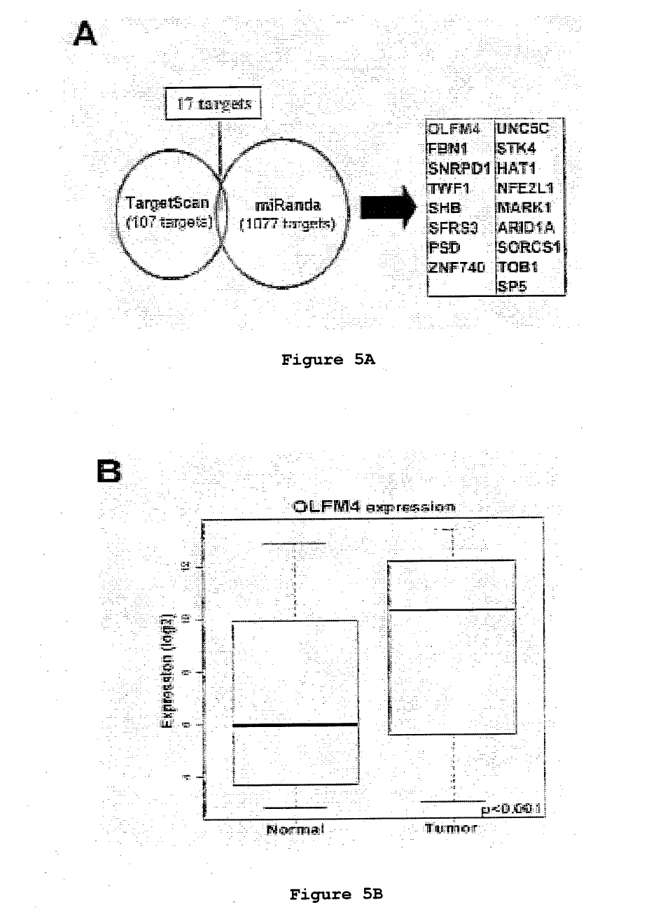

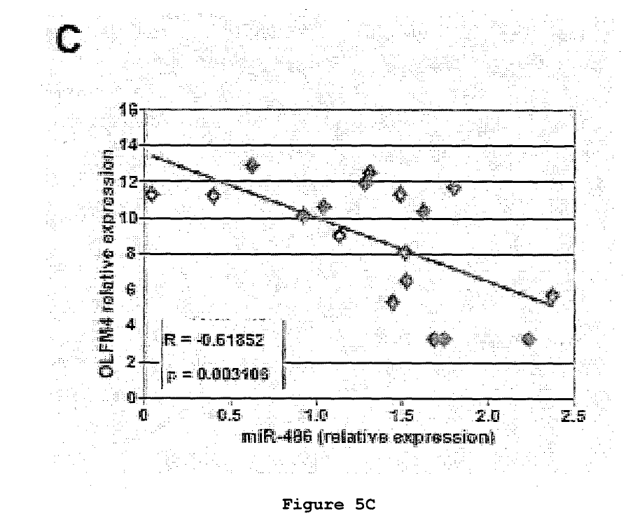

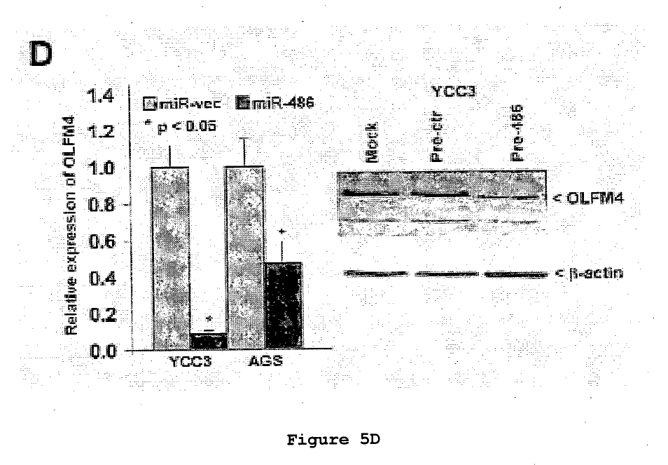

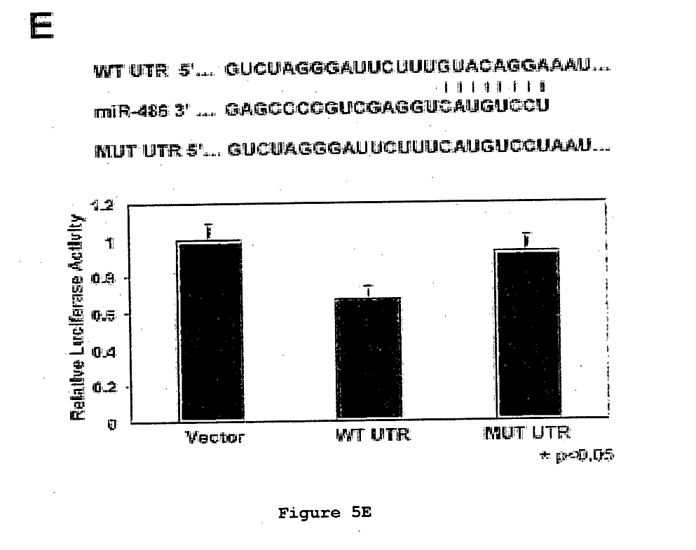

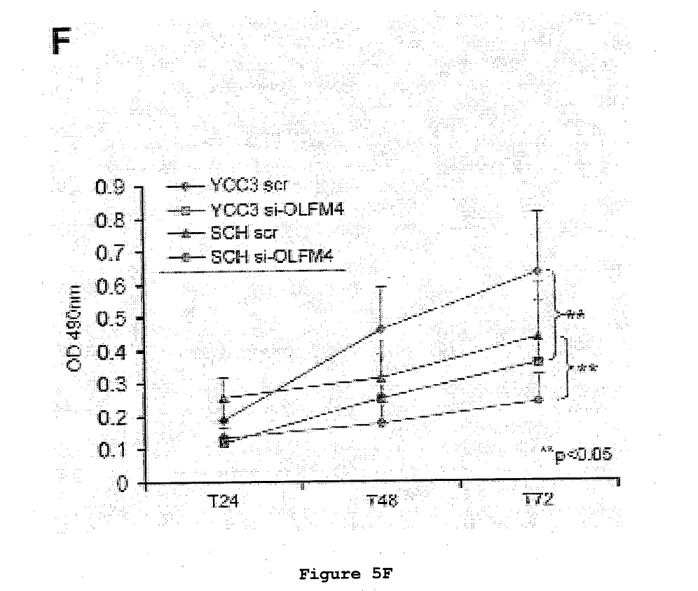

FIG. 5 shows the direct regulation of Olfactomedin-4 (OLMF4) by miR-486. FIG. 5 comprises FIGS. 5A-5E. FIG. 5A shows miR-486 target prediction using miRanda v.30 and TargetScan 5.1 algorithms. Seventeen (17) targets were common to both prediction programs (see listings in table in the right panel of FIG. 5A, where OLFM4 has been highlighted). FIG. 5B shows that OLFM4 is highly expressed in primary gastric tumors compared to matched normal tissues (p<0.001). FIG. 5C shows OLMF4 and miR-486 in intestinal-type primary gastric cancers and matched normals. A significant negative correlation (R=0.61852; p=0.003106) of miR-486 to OLMF4 expression was observed in intestinal-type gastric cancers and 7 matched normal tissues analyzed. FIG. 5D shows that OLFM4 protein levels are regulated by miR-486. Western blot analyses of OLFM4 protein in cells transfected with miR-486 or negative control mimics. OLFM4 protein levels were lower in YCC3 cells expressing miR-486 as compared to control-miR expressing cells. Similar reductions the OLFM4 proteins were also observed in miR-486 expressing AGS cells. Quantitative real-time PCR showed that OLFM4 transcript is also reduced in cells transfected in miR-486 as compared to controls. (* denotes the t-test for OLFM4 expression in miR-486 vs control cells). FIG. 5E shows that OLFM4 is a direct target of miR-486. The predicted miR-486 target region found in the OLFM4 mRNA 3′UTR was cloned downstream of luciferase in a pMIR-Report-luciferase reporter vector. Reporter constructs were co-transfected with miR-486 and negative control mimic molecules into AGS cells. Luciferase reporter assays were normalized to β-galactosidase activities and experiments were performed in triplicates. Data were plotted after normalized against the negative control mi-RNA mimics (columns represent means; bars represent standard deviations; *, p<0.05). FIG. 5F shows that silencing of OLFM4 in gastric cancer cells YCC3 and SCH by si-RNA reduces cell proliferation capacity. YCC3 and SCH cells were transfected with si-RNAs against OLFM4 or scrambled controls (scr, negative control). Experiments were performed in triplicates. Y-axis denotes the (absorbance at 490 nm (cell proliferation) and x-axis is the assay time-points (points represent means; bars represent standard deviations; significance for the difference in growth in denoted by **, p<0.05).

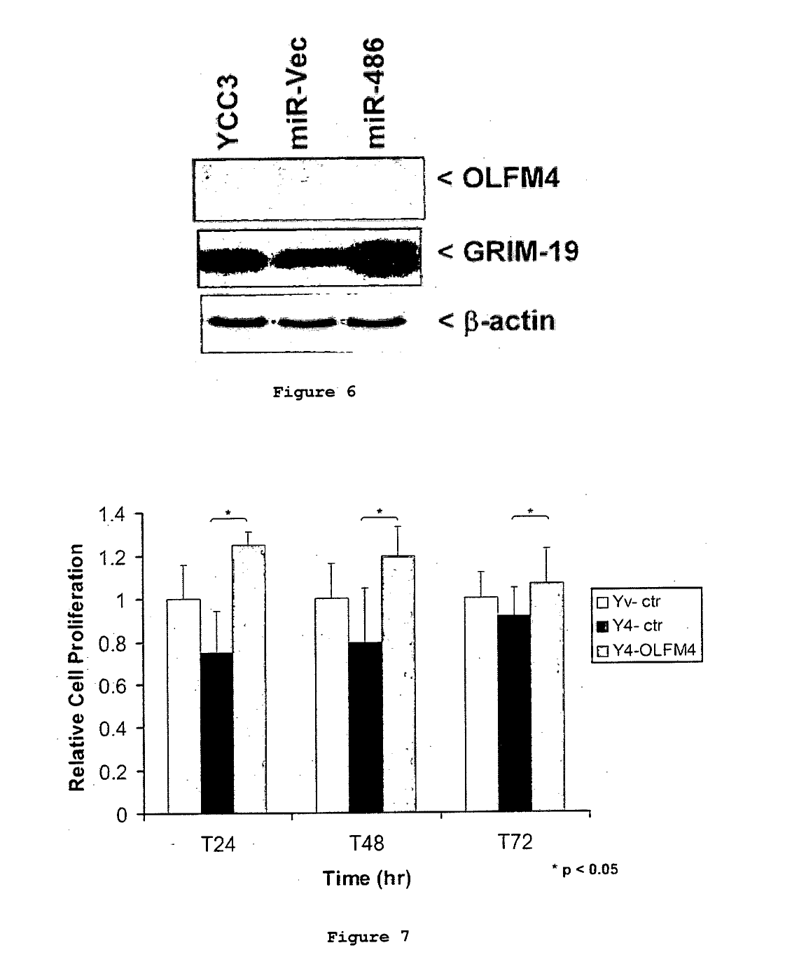

FIG. 6 shows the downregulation of OLFM4 protein by miR-486 induces protein upregulation of GRIM-19. In this Figure, a gastric cell line comprising YCC3 cells were used to generate stable cells expressing miR-486 or empty vector control. The selected stable cells were then analyzed for the protein levels of OLFM4 and GRIM-19.

FIG. 7 shows OLFM4 overexpression counteracts the growth inhibition effects of miR-486. To obtain this Figure, gastric line YCC3 cells stably expression miR-486 (Y4) were transfected with negative control (ctr) or OLFM4 expression plasmids. Transfected cells were assayed for proliferation at 24, 48 and 72 hr post-transfection. Triplicate experiments were performed for each set. Data were normalized to the proliferation of YCC3 cells stably expressing empty vector (Yv) (columns represent means; bars represent standard deviation; significance for the difference in growth is denoted by *, p<0.05).

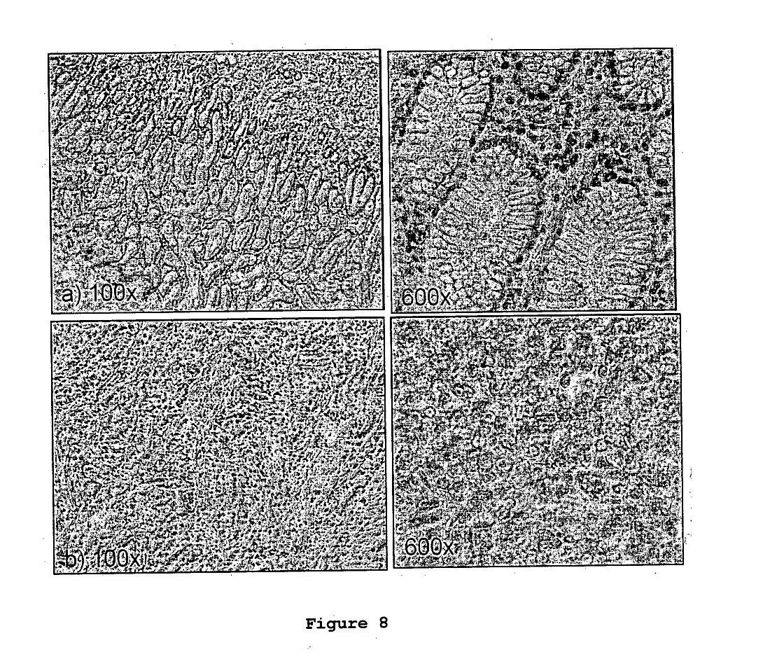

FIG. 8 is obtained by in-situ hybridization on FFPE gastric tissue sections (normal and with tumor) using the double-DIG labeled mercury LNA miR-486-5p detection probes. FIG. 8a) displays normal gastric epithelium showing miR-486 expression at low power (×100) and high power (×600) magnification. FIG. 8b) displays the matching gastric epithelium with no miR-486 expression being observed (×100 and ×600 magnification).

### EXAMPLES

Non-limiting examples of the embodiments will be further described in greater detail by reference to specific Examples, which should not be construed as in any way limiting the scope of the invention.

SEQUENCE LISTINGS

SEQ ID NO: 1 is hsa-miR-486 (miR-486) as follows,
which has the same sequence as the mature form
of miR-486 (5′-3′):
UCCUGUACUGAGCUGCCCCGAG
SEQ ID NO: 2 is a precursor of miR-486 (5′-3′):
GCAUCCUGUACUGAGCUGCCCCGAGGCCCUUCAUGCUGCCCAGCUCGGG
GCAGCUCAGUACAGGAUAC
SEQ ID NO: 3 is OLFM4 UTR (5′-3′):
GUCUAGGGAUUCUUUGUACAGGAAAU
SEQ ID NO: 4 is miR-486 (3′):
GAGCCCCGUCGAGGUCAUGUCCU
The binding motif of miR-486 for binding to the UTR of OLFM4 has the following core nucleotide sequence (5′-3′): UCCUGUAC

### Materials and Methods

### Primary Gastric Cancer Samples and Cell Lines

Primary gastric tumors and adjacent matched normal gastric tissues were obtained from the National Cancer Centre Singapore and the Singhealth Tissue Repository. Primary samples were collected with signed patient informed consent and with approval from institutional review boards. gastric cancer cell lines AGS, Kato III, SNU1, NCIN87, and Hs746T were obtained from the American Type Culture Collection and AZ-521, TMK1, MKN1, MKN7, MKN45 cells were obtained from the Japanese Collection of Research Bioresources. SCH cells were a gift from Yoshiaki Ito (Cancer Sciences Institute of Singapore). YCC1, YCC3, YCC6, YCC7 cells were a gift from Sun-Young Rha (Yonsei Cancer Center, South Korea). All cell lines were tested and authenticated by the respective cell line bank (ATCC, JCRB) or originating institution (YCC) by several methods including DNA fingerprinting and/or cytogenetics. For the following Examples, the cell lines were re-authenticated by comparing their genome-wide copy number (array-CGH) and mutational profiles to the published literature.

mi-RNA Expression Profiling

Total RNA was extracted from primary tissues and cell lines using the miRVana mi-RNA Isolation Kit (Ambion, Inc, Austin, Tex., USA) according to the manufacturer's instructions. RNA samples were hybridized to Agilent Human mi-RNA Microarrays (V2) representing 723 human and 76 human viral mi-RNAs, and scanned using an Agilent DNA Microarray Scanner (Model G2565BA). mi-RNA expression values were normalized against background signals using Feature Extraction Software (Agilent). The mi-RNA data was also subjected to a log 10 transformation followed by 7 median centering across probes, prior to in-depth analysis. The mi-RNA expression data has been deposited into GEO under accession number GSE23739.

### Microarray Data Analysis

Differentially expressed mi-RNAs were identified using the Significance Analysis of Microarrays (SAM) program in BRB-ArrayTools using a False Discovery Rate (FDR) cutoff of <0.01. Significance of Pearson correlations (R) between two N-element vectors were estimated from the Student t-distribution, against the null hypothesis that the observed value of t=R/√[(1−R2)/(N−2)] arises from a population in which the true correlation coefficient is zero. Of 146 differentially expressed mi-RNAs initially identified by SAM (FDR<0.01), the subsequent analysis focused on the top 40 mi-RNAs exhibiting the highest positive log fold-change (most upregulated) and the top 40 mi-RNAs with the lowest negative log fold-change (most downregulated). Among the 40 most significant downregulated mi-RNAs in tumors, 16 candidate tumor suppressor mi-RNAs were triaged provided that those mi-RNAs met the additional criterion of detectable expression in an independent normal stomach sample from a healthy patient (Stratagene, La Jolla, Calif.), at levels greater than the median expression in gastric cancer cell lines.

### Real-Time Quantitative Reverse Transcription-PCR (RT-qPCR)

RT-qPCR was performed using a ABI7900HT Fast real-time PCR system (Applied Biosystems, Foster City, Calif.). TaqMan® Universal PCR Master Mix (Applied Biosystems), TaqMan® Reverse Transcription Kit and TaqMan® MicroRNA Assay kits (Applied Biosystems) were used to measure miR-486 expression levels according to the manufacturer's instructions. Each PCR was normalized against an RNU6B internal control. All PCR reactions were performed in triplicate.

mi-RNA Transfections

Gastric cancer cells were transfected with precursor molecules mimicking miR-486 (Ambion) or scrambled sequence miRNAs (Pre-miR negative control #1, Ambion) using LipoFectamine 2000 (Invitrogen), according to the manufacturer's instructions. For stable transfections, a 700 bp fragment containing the miR-486 mature sequence was amplified from genomic DNA and cloned into the mirVec vector. These constructs were transfected into GC cell lines using Superfect transfection reagent (Qiagen). Pooled clones stably expressing either the empty vector or miR-486 were obtained by blasticidine selection. For mi-RNA silencing, an inhibitor of miR-486 or negative inhibitor control (Ambion) were transfected into the YCC6 cells using HiPerFect transfection reagent (Qiagen) according to the manufacturer's instructions.

### Si-RNA Silencing and Overexpression of OLFM4

YCC3 and SCH cells were transfected with ON-TARGETplus si-RNAs against OLFM4 (Dharmacon) or non-targeting si-RNA controls (si-SCR) using Lipofectamine 2000) (Invitrogen according to the manufacturer's instructions and re-transfected after 24 hours. 5 hours after the second transfection, the cells were trypsinised, counted and plated into 96-well dishes for proliferation assays at 24 h, 48 h and 72 h time points. Each experiment was repeated three independent times. For overexpression of OLFM4 in YCC3 stable cells, cells were transfected with expression plasmid for human OLFM4 (OriGene) using SuperFect reagent (Qiagen). Cells were counted and plated into 96-well dishes for proliferation assays as above.

### Cell Proliferation and Anchorage Independent Growth Assays

Cell proliferation assays were performed using a CellTiter96 Aqueous non-radioactive Cell Proliferation Assay kit (Promega, Madison, Wis.) following the manufacturer's instructions and measured using a PerkinElmer plate reader (EnVision™ Multilabel Plate Reader). Triplicate assays were performed. To assess anchorage independent growth, 1.5 mL of 0.5% agar (BD Bioscience) supplemented with complete culture medium and 10% FBS were layered into sixwell plates as bottom agar. Ten thousand cells mixed with 1.5 mL of 0.35% agar supplemented with complete culture medium were layered onto the solidified bottom agar. Plates were incubated at 37° C. in a humidified incubator with 5% CO2 for 2-3 weeks, stained with thiazolyl blue tetrazolium bromide, and scanned. Colonies were quantified using the Image J program (http://rsbweb.nih.gov/ij/). Experiments were carried out in triplicate.

### Cell Migration and Invasion Assays

Cell migration assays were performed using Biocoat™ 24-well chambers with 8-μm pore filter inserts (BD Bioscience). After chamber rehydration, 5×104 cells were transferred to the upper chamber in 500 μl serum-free medium. Complete medium with 10% FBS was used as a chemoattractant. Cells were allowed to incubate for 48 hours, and migrated cells on the lower surface of insert or in the wells were trypsinized and their cell numbers counted. Each assay was performed in triplicate, and the results were averaged over three independent experiments. Cell invasion assays was done similarly using Biocoat™ Matrigel™ invasion chambers with 8-μm pore polycarbonate membranes precoated with Matrigel™ Matrix (BD Biosciences).

### Array-CGH Assays and Copy Number Analysis

One hundred and six (106) primary gastric tumors were profiled using Agilent Human Genome 244A CGH Microarrays (Agilent Technologies, Santa Clara, Calif.) containing approximately 240,000 distinct 60-mer oligonucleotide probes spanning the entire human genome. Sample labeling and hybridizations were performed according to the manufacturer's instructions. Hybridized slides were scanned on an Agilent DNA Microarray Scanner (Agilent Technologies) and images were extracted using Agilent Feature Extraction software. Copy number variation levels for the miR-486 locus were analyzed using Nexus software (http://www.biodiscovery.com/index/nexus).

miRNA Target Prediction.

Predicted miR-486 gene targets were identified using both the miRBase (http://www.mirbase.org/) and TargetScan (http://www.targetscan.org/) algorithms. Targets commonly predicted by both algorithms were nominated for further analysis.

### Western Blotting

Cell pellets were lysed in ice-cold lysis buffer (50 mM Tris-HCl, pH 8.0, 300 mM NaCl, 1 mM EDTA, pH 8, 0.5 1% NP-40, 10% glycerol, 1 mM DTT, 0.2 mM PMSF supplemented with protease inhibitor cocktail and phosphatase inhibitor cocktail). After centrifugation at 14,000 rpm for 20 min at 4° C., supernatants were collected, and protein concentrations determined using a RC-DC protein assay kit (Biorad, USA). Protein extracts were separated on 12% SDSpolyacrylamide gels and transferred to PVDF membranes. OLFM4 (Sigma and Santa Cruz) and β-actin (Chemicon) primary antibodies were used, along with corresponding secondary antibodies conjugated with DyLight 680/800 (Thermo Scientific). Western blots were scanned using a LI-COR Odyssey IR Imager.

### OLFM4 3′UTR Luciferase Reporter Assays

A 400 bp section of the OLFM4 3′UTR (primer F: 5′-atcgacgcgtcactagagatctaggacat-3′, primer R: 5′-atcgacgcgttagaatatataagcatgcc-3′) containing the predicted miR-486 binding sequence was PCR amplified and cloned into the MluI site in the luciferase reporter pMIR-Report vector (Ambion). Mutant reporter constructs where the miR-486 binding sites were mutated were generated by PCR-based mutagenesis. AGS were co-transfected with a) 0.5 ug of pMIR-Report vectors containing either the wild-type or mutated OLFM4 3′UTR, and b) 37.5 nM negative control precursor molecules or miR-486 precursor molecules using Attractene (Qiagen). The pMIR-Report-β-GAL plasmid containing the β-galactosidase gene (0.2 ug) was also included as an internal control for transfection efficiency. Cells were harvested 48 h after transfection, and analyzed for luciferase activity using the Luciferase reporter assay system and β-galactosidase assay system (Promega). Luciferase activity was normalized relative to β-galactosidase activity. Triplicate experiments were performed.

### In-Situ Hybridization (ISH)

Double DIG miRCURY LNA micro-RNA probes (Exigon, Vedbaek, Denmark) were used to detect the expression of mir-486 in gastric FFPE sections. Tumor and matched normal tissues were deparaffinized and rehydrated using alcohol gradients. miRNAs were demasked using Proteinase-K to allow the double DIG-labeled LNA mir-486-5p (50 nm) probe to hybridize to the micro-RNA sequence. Hybridization was performed at 55° C. for 1 hr followed by incubation at room temperature for 0.1 hr with sheep anti-DIG antibody (1:800 dilution) conjugated with Alkaline Phosphatase (AP). Freshly prepared AP substrate [4-nitro-blue tetrazolium (NBT) and 5-bromo-4-chloro-3′-indolylphosphate (BCIP)] was applied to the sections and incubated at 30° C. for 2 hrs. The reaction was stopped by KTBT buffer (50 mM, Tris-HCL, 150 mM NaCl and 10 mM KCL) and counterstained using nuclear fast red. The slides were then mounted with Eukitt mounting medium and analyzed using light microscopy.

### Example 1

### Identification of Differentially Regulated mi-RNAs in Primary Gastric Tumors

To identify the micro RNAs which exhibit expression changes in gastric cancer, 40 gastric tumors and 40 non-cancerous gastric tissues were profiled on Agilent mi-RNA microarrays by measuring ˜800 mi-RNAs (723 human and 76 viral miRNAs). Gastric tumors are traditionally classified by histology into 2 major groups—intestinal (expanding, or differentiated), and diffuse (infiltrative or undifferentiated). This analysis covered both gastric cancer subtypes, since half of the tumors were intestinal-type gastric cancer (20 samples), while the other half were diffuse-type cancers. An initial unsupervised analysis where the gastric samples were clustered using mi-RNAs detectably expressed in ≧25% of the samples (329 probes) resulted in an overall partitioning of gastric tumors away from normals, indicating the existence of pervasive miRNA expression differences between the two groups (Fig. S1 not shown). The subsequent analysis focused on mi-RNAs exhibiting striking differences in expression between gastric normal and tumor tissues. Using Significance Analysis of Microarrays (SAM), 80 mi-RNAs were identified, which were significantly differentially expressed between the two groups (FDR<0.01) as shown in FIG. 1A. The 80 mi-RNAs, which were either upregulated or downregulated, may modulate (e.g. decrease or increase) the expression of an oligonucleotide encoding for a protein that may function in cancer. Table 1 below provides a list of the top 80 miRNAs that were upregulated or downregulated in gastric tumor tissues. Referring to the list of the downregulated mi-RNAs in Table 1, it should be noted that a mi-RNA such as hsa-mir-486-5p may have a binding motif with a core nucleotide sequence of UCCUGUAC, while another mi-RNA such as hsa-miR-1229 may target the 3′ untranslated region of the oligonucleotide encoding for a protein selected from the group consisting of OLFM4, SP5, TOB1, ARID1A, FBN1 and HAT1. The exemplary mi-RNA has-miR-1229 may include a nucleotide sequence being complementary to the nucleotide sequence of the 3′untranslated region of the oligonucleotide encoding for a protein.

TABLE 1
Eighty (80) mi-RNAs which exibited striking
differences in expression between gastric normal and
gastric tumor tissues. The 80 mi-RNAs are separated into
two groups depending on their upregulation or down
regulation in gastric tumor tissues (see following page).

| miRNA that are up- | miRNA that are |
|---|---|
| regulated in gastric | down-regulated in |
| tumors | gastric tumors |
| hsa-miR-21 | hsa-miR-623 |
| hsa-let-7i | hsa-miR-134 |
| hsa-miR-16 | hsa-miR-181c(a) |
| hsa-miR-20a | hsa-miR-654-5p |
| hsa-let-7f | hsa-miR-936 |
| hsa-miR-199b-3p | hsa-miR-939 |
| hsa-miR-214 | kshv-miR-K12-3 |
| hsa-miR-15a | hsa-miR-550 |
| hsa-miR-17 | hsa-miR-486-5p |
| hsa-miR-142-3p | hsa-miR-575 |
| hsa-let-7a | hcmv-miR-UL70-3p |
| hsa-let-7d | hsa-miR-638 |
| hsa-miR-18a | hsv1-miR-H1 |
| hsa-miR-27a | hsa-miR-139-3p |
| hsa-miR-146b-5p | hsa-miR-202 |
| hsa-miR-192 | hsa-miR-378 |
| hsa-miR-23a | hsv1-miR-LAT |
| hsa-let-7e | hsa-miR-596 |
| hsa-miR-107 | hsa-miR-188-5p |
| hsa-miR-103 | hsa-miR-28-3p |
| hsa-miR-19a | hsa-miR-30d |
| hsa-miR-199b-5p | hsa-miR-572 |
| hsa-miR-451 | hsa-miR-1225-5p |
| hsa-miR-200a | hsa-miR-345 |
| hsa-miR-27b | hsa-miR-30a |
| hsa-miR-223 | hsa-miR-671-5p |
| hsa-miR-18b | hiv1-miR-H1 |
| hsa-miR-29b | hsa-miR-148a(a) |
| hsa-miR-224 | hsa-miR-222 |
| hsa-let-7g | hsa-miR-10b(a) |
| hsa-miR-135b | hsa-miR-564 |
| hsa-miR-200b | hsa-miR-193b(a) |
| hsa-miR-34a | hsa-miR-125a-3p |
| hsa-miR-199a-5p | hsa-miR-370 |
| hsa-miR-125b | hsa-miR-375 |
| hsa-miR-215 | hsa-miR-923 |
| hsa-miR-885-5p | hsa-miR-513c |
| hsa-miR-331-3p | hsa-miR-513a-5p |
| hsa-miR-28-5p | hsa-miR-494 |
| hsa-miR-301a | hsa-miR-513b |

The differentially regulated mi-RNAs were divided into three distinct classes based on their global expression patterns. The first class of mi-RNAs exhibited high expression in tumors relative to normals—these may represent potential prooncogenic mi-RNAs contributing to gastric cancer development and progression. It has been accordingly observed that among the most highly expressed mi-RNAs in this class were miR-21, miR-27a and miR-17, three mi-RNAs that are known to exert oncogenic functions in multiple cancer types including gastric cancer. The re-identification of these known oncogenic mi-RNAs supports the biological validity of the mi-RNA microarray data. Further observed is the upregulation of hsa-miR-16 and has-miR-214 in gastric cancer, two mi-RNAs known to be down-regulated in prostate and cervical cancers. It is thus possible that certain mi-RNAs may exert either pro or anti-oncogenic functions that are dependent on tissue type.

The second class comprised mi-RNAs exhibiting downregulation in tumors relative to normals. Such mi-RNAs might represent candidates for potential tumor-suppressor mi-RNAs. Given the relative lack of validated tumor suppressor mi-RNAs in gastric cancer compared to oncogenic mi-RNAs, mi-RNAs in the second class were further analysed. Using multiple filtering criteria, a set of 16 candidate tumor-suppressor mi-RNAs were nominated (see FIG. 1B). Among the 16 candidates, miR-375 was identified, which is a known tumor suppressor mi-RNA in gastric cancer. The 16 candidate tumor-suppressor mi-RNAs are further considered in the next Example:

A third and smaller class of mi-RNAs was detected. These mi-RNAs were differentially expressed between intestinal and diffuse-type gastric cancers (see FIG. 1A, bottom panel). In general, these mi-RNAs (hsa-let7d*, hsa-miR-328, hsa-miR-32*, hsa-miR-1227, hsa-miR-206, hsa-miR-1229, hsa-miR-595 and hsa-miR-631) were largely downregulated in diffuse type gastric cancer compared to intestinal-type gastric cancers. These results raise the possibility that differences in mi-RNA expression may also exist between the two major histologic subtypes of gastric cancer.

### Example 2

### Expression Patterns of miR-486, a Candidate Tumor Suppressor Mi-RNA in Gastric Cancer

A handful of the 16 nominated mi-RNAs exhibiting decreased expression in gastric cancer were selected for functional validation to determine whether the decreased expression in gastric cancer would represent bona-fide gastric cancer tumor suppressors. This particular Example reports the detailed findings for the candidate miR-486 that was validated.

miR-486 is located on chromosome 8p11 within intron 41 of the Ankyrin-1 (Ank1) gene (see FIG. 2A). miR-486 is transcribed from an alternative promoter within intron 40 of the Ankyrin-1 gene, and has been previously shown to regulate PI3K signaling in muscle cells by targeting the PTEN gene. In contrast to its role in muscle development, the role of miR-486 in epithelial tumorigenesis is currently unclear. Three observations suggest that miR-486 may play a potential tumor suppressive role in gastric cancer. First, besides exhibiting reduced expression in tumors compared to the normal tissues (see FIG. 8), miR-486 is expressed in a strikingly reciprocal pattern to the oncogenic mi-RNAs miR-17, miR-21 and miR-27a (see FIG. 2B, lower panel). Second, to validate the microarray results, reverse-transcription quantitative PCR (RT-qPCR) was performed to directly measure miR-486 expression levels in a cohort of 28 primary gastric cancer tissues and matched adjacent normal tissues. 80% of the tumors expressed decreased levels of mir-486 expression by at least two fold compared to matched normal tissues, confirming that miR-486 is expressed at significantly lower levels in gastric cancers (p<0.05, paired t-test) (see FIG. 2C). Third, to further validate the reduced expression of miR-486 in gastric cancer cells, the expression of mir-486 was investigated in a panel of 15 gastric cancer cell lines. Similar to the primary tumors, fourteen out of fifteen gastric cancer cell lines underexpressed miR-486 while simultaneously overexpressing the miR-17 oncogenic mi-RNA (p=0.001; see FIG. 2D). Taken collectively, these results suggest that miR-486 may play a tumor-suppressive role in gastric cancer.

### Example 3

### miR-486 Expression Inhibits Proliferation, Anchorage Independent Growth, Migration and Invasion in Gastric Cancer Cells

Three gastric cancer cell lines (YCC3, AGS and SCH) were selected in further studies to investigate the functional significance of miR-486 downregulation in gastric cancer, as these three lines express low levels of miR-486 (see. FIG. 2D). Synthetic mir-486 precursor molecules were transfected into these cells lines to restore mir-486 expression, and restoration of miR-486 expression in these cells was confirmed by qRT-PCR (see FIG. 3A). First, the cell proliferation rates were compared between the control and mir-486 transfected cells at various time points. In all three cell lines, the growth of miR-486 transfected cells was significantly reduced compared to cells transfected with negative-control miRs (see FIG. 3B). This result suggests that restoring miR-486 expression is sufficient to inhibit cellular proliferation in gastric cancer.

Second, to examine the importance of miR-486 in the tumorigenesis of gastric cancer cells, anchorage-independent growth assays were performed. Using a blasticidin selection protocol, stable pools of SCH, YCC3 and AGS cells expressing miR-486 or empty vector controls were generated. SCH and YCC3 cells transfected with empty vector controls grew well in soft agar, forming distinct colonies (see FIG. 3C). In contrast, SCH and YCC3 cells expressing miR-486 exhibited a dramatic reduction in the number of soft agar colonies (FIG. 3C), demonstrating transforming abilities less than 50% of the control cells. Similar data was obtained for AGS cells (FIG. 3C, right panel). These results suggest that miR-486 can suppress the tumorigenecity of gastric cancer cells in vitro.

Third, to assess the effect of mir-486 in gastric cancer migration and invasion, AGS cells stably expressing mir-486 or empty vectors were tested. AGS cells expressing vector controls migrated robustly in Transwell assays (see FIG. 3D, top), while AGS cells overexpressing mir-486 exhibited a significant reduction in migration capacity (p<0.05, about 2-fold). Similarly, in invasion assays, AGS cells overexpressing miR-486 exhibited a 2-fold reduced capacity for invasion compared to controls (p=0.05) (see FIG. 3D, bottom).

Fourth, of the 15 gastric cancer cell lines tested, one line (YCC6) was found to express above-average levels of miR-486 (see FIG. 2D). To investigate the cellular effects of silencing rather than overexpressing miR-486, endogenous miR-486 expression was inhibited in YCC6 cells by transfecting the cells with miR-486 inhibitors (anti-mir-486). Efficient inhibition of mir-486 expression was confirmed by qRT-PCR (see FIG. 3E). miR-486-suppressed YCC6 cells exhibited a modest but significant enhancement of cell proliferation compared to control transfected cells (p<0.05, FIG. 3E). Taken collectively, these results indicate that restoring miR-486 expression is sufficient to suppress several pro-oncogenic traits in vitro, while conversely suppressing miR-486 expression is sufficient to enhance such traits, consistent with miR-486 playing a tumor suppressive role in gastric cancer.

### Example 4

### Frequent Genomic Loss of miR-486 in Gastric Cancer

Besides epigenetic silencing, genomic loss is another mechanism by which tumor suppressor mi-RNAs can be downregulated in cancer. Notably, miR-486 is located on chromosome 8p11, a frequent region of loss-of-heterozygosity in many cancers, including gastric cancer. While ANK1, the gene within which miR-486 lies, has been proposed as a potential tumor-suppressor gene in this region, there is in reality very little functional evidence supporting an anti-oncogenic role for ANK1. These findings raise the possibility that other genetic elements on 8p11 14 lying close to ANK1, such as miR-486, may represent important driver elements for the frequent genomic losses associated with this region in cancer.

To investigate if miR-486 might be genomically deleted in gastric cancer, an in-house array-CGH database of 106 gastric tumors profiled on Agilent 244K microarrays was analyzed. This analysis affirmed genomic loss of the miR-486 locus within a window of 500 kb on chromosome 8 in 25-30% of gastric cancers (see FIG. 4A). In individual gastric cancers, the size of the genomic deletion ranged from 100 kb to 300 kb. Moreover, arguing against the possibility of another nearby gene driving these deletions, two tumors (gastric cancer GC990187 and GC200088) exhibited highly focal genomic regions specifically deleting the miR-486 locus (GC990187—chr8:41,582,276-41,727,172 (135 kb); GC200088—chr8:41,582,276-41,717,096 (145 kb)) (see FIGS. 4B and 4C), while retaining a portion of the ANK1 gene. A genomic deletion at the miR-486 region in the gastric cancer cell line SCH has also been observed (data not shown). These results indicate that the miR-486 genomic locus is frequently deleted in gastric cancers, supporting the notion that miR-486 is a tumor suppressor miRNA.

### Example 5

### The Anti-Apoptotic Factor OLFM4 is a Direct miR-486 Target

To better understand the mechanisms underlying the tumor suppressive capacities of miR-486, candidate miR-486 target genes with potential pro-oncogenic functions were searched. Using two mi-RNA target prediction programs (TargetScan 5.1 and miRanda v3.0), 17 candidate miR-486 target genes commonly predicted by both programs were identified (see FIG. 5A). Among these 17 genes, two genes (TOB1 and ARID1A) have been previously shown to be associated with tumor suppressive functions. More importantly, genes SP5 and OLFM4 were identified as potential prooncogenic miR-486 target genes. Attention was specifically drawn to OLFM4 (Olfactomedin-4/GW112), as OLFM4 has been previously reported to be highly expressed in gastric tumors compared to normal tissues. Both prediction programs identified one potential miR-486 binding site in the OLFM4 3′UTR.

Functionally, OLFM4 has also been shown to behave as an anti-apoptotic factor and to promote tumor growth and invasion. This Example found that OLFM4 was indeed highly expressed in gastric cancers compared to gastric normals (p<0.001) (see FIG. 5B). Also supporting recent findings, the high expression of OLFM4 in tumors was largely associated with intestinal-type gastric cancers (see Table 2 below).

TABLE 2
High OLFM4 Expression is associated with
intestinal-type gastric cancer.

| Lauren's | | | | |
|---|---|---|---|---|
| Classification | Intestinal | Diffuse | Mixed | Total |
| OLFM4 low expression | 37 | 42 | 8 | 87 |
| OLFM4 high expression | 52 | 24 | 11 | 87 |
| Total | 89 | 66 | 19 | 174 |

A Pearson chi-squared statistic was used to test the association of the expression of OLFM4 with gastric cancer histological subtype (174 tumors). High OLFM4 expression was positively associated with intestinal-type gastric cancer (Pearson chi-square test, p=0.019).

To investigate relationships between endogenous miR-486 and OLFM4 expression, 11 intestinal-type primary gastric cancers and matched non-malignant tissues for which both miR-486 and OLFM4 expression were available were analyzed. miR-486 was significantly negatively correlated to OLFM4 expression (R=−0.619, p=0.0031) (see FIG. 5C), consistent with miR-486 targeting OLFM4 in vivo. To directly assess the functional impact of miR-486 on OLFM4 expression, gastric cancer cells (YCC3 and AGS) were transfected with miR-486 precursors and measured endogenous OLFM4 expression levels. Restoration of miR-486 in YCC3 and AGS cells caused a decrease in OLFM4 protein and transcript levels, relative to control-transfected cells (see FIG. 5D). These results indicate that restoration of miR-486 in gastric cancer cells is sufficient to suppress OLMF4 expression. Luciferase reporter assays were also performed to demonstrate a direct functional role of the predicted miR-486 binding sites in the OLFM4 3′-UTR. Luciferase reporters were constructed containing either a wild-type OLFM4 3′-UTR sequence containing the miR-486 binding site (WT-UTR), or a mutated OLFM4 3′UTR where the miR-486 seed sequence binding sites were altered (MUT-UTR)(see FIG. 5E). The WT-UTR and MUT-UTR luciferase reporter constructs were transfected into gastric cancer cells, along with miR-486 or negative control mi-RNAs. Luciferase expression of the WT-UTR reporter was significantly decreased compared to MUTUTR (p<0.05) or vector-expressing cells in a miR-486-dependent manner, indicating that miR-486 is able to reduce the reporter activity of WT-UTR but not MUT-UTR (see FIG. 5E). This result strongly indicates that that miR-486 directly targets the OLFM4 3′UTR, resulting in the translation inhibition of OLFM4 protein.

OLMF4 Expression Promotes Gastric Cancer Cell Proliferation and Inhibits the Anti-Oncogenic Effects of miR-486

OLFM4 has been proposed to promote tumor growth by functioning as an anti-apoptotic protein attenuating the apoptotic function of GRIM-19, a celldeath regulatory protein. To establish a functional role for OLFM4 in gastric cancer, OLFM4 was silenced in YCC3 and SCH cells and cell proliferation assays were then conducted. OLFM4-silenced YCC3 and SCH cells exhibited significantly slower cellular proliferation compared to control si-RNA treated cells (see FIG. 5F). This result suggests that OLFM4 activity may be required for gastric cancer development and progression. It has been observed that downregulation of OLFM4 by miR-486 also resulted in up-regulation of GRIM-19, the proposed target of OLFM4 (see FIG. 6).

Finally, to establish if the anti-proliferative effects caused by miR-486 restoration might depend, at least in part, on suppression of OLFM4 activity, rescue experiments were performed where YCC3 cells stably expressing miR-486 (Y4 cells) were transfected with OLMF4 expressing constructs. It is found that Y4 cells overexpressing OLMF4 exhibited a significant increase in cell proliferation compared to control Y4 cells, comparable to parental YCC3 cells (see FIG. 7). These results suggest that the tumor-suppressive effects of miR-486 are likely to be mediated, at least in part, through its effect on OLFM4 activity. In summary, the data suggests that miR-486 is a tumor suppressor in gastric progression, that its down-regulation in gastric cancer by genomic deletion may facilitate tumor growth, in part by causing OLFM4 upregulation.

In general, referring to Examples 1-5, recent evidence has convincingly shown an important role for mi-RNAs in many human cancers. In gastric cancer, for example, previous mi-RNA profiling studies have led to the collective identification of approximately −80 mi-RNAs exhibiting dysregulated expression between tumors and normals. However, beyond their observed expression patterns, relatively few tumor suppressor miRNAs have been functionally explored in gastric or other forms of cancer, notable exceptions being miR-375 (regulating PDK1 and 14-3-3ζ), and miR-141, whose downstream targets are still unclear. In one embodiment described herein, miR-486 has been administered to be a candidate gastric tumor suppressor. Restoration of miR-486 in multiple gastric cancer cell lines significantly reduced several pro-oncogenic traits, including cell proliferation, anchorage independent growth, and cell migration/invasion, while silencing of miR-486 in YCC6 cells enhanced proliferation. It is worth noting that it was reported that miR-486 is under-expressed in several other cancer types besides gastric cancer and miR-486 has also been reported to be downregulated in glioblastoma stem cells. Thus, the tumor suppressive role of miR-486 in cancer is not limited to gastric cancer alone but it is relevant also to other cancer types.

It was found that about 25-30% of gastric tumors, exhibited a genomic loss of the chromosome 8p11 region where miR-486 is located. This frequency of 8p loss is comparable to previous array-based CGH studies of gastric cancer. Moreover, genomic deletions in miRNAs have been reported as a mechanism for miRNA downregulation, as shown for miR-101, miR-15a, and miR-16-1. However, because 70% of gastric cancers did not exhibit observable genomic loss of miR-486, genomic deletions alone are unlikely to fully explain the pervasive downregulation of miR-486 in gastric cancer. It is thus almost Certain that other gastric cancer tumors must employ alternative mechanisms to achieve miR-486 downregulation, such as for example epigenetic silencing or transcriptional repression, as reported for miR-124 or transcriptional suppression of the miR-29a promoter by myc. Evidence of miR-486 epigenetic regulation when gastric cancer cell lines were treated with either inhibitors of DNA methyltransferanse or histone acetyltransferases was not observed.

It was demonstrated that OLFM4 is a direct target gene of miR-486. However, it is likely over-simplistic to expect that the anti-oncogenic effects of miR-486 can be entirely explained by its ability to regulate a single gene alone, particularly since previous studies investigating the Cellular functions of miRNAs have shown that a single miRNA can often regulate many genes and gene targets. Besides OLFM4, in bio-informatic analysis, 16 other potential miR-486 target genes were identified, several of which may function in cancer (eg FBN1, HAT1, SP5, TOB1, ARID1A and OLFM4), and miR-486 has also been shown to target PTEN in muscle cells. Nevertheless, OLFM4 is a biologically relevant miR-486 target in the context of gastric cancer, for example. OLFM4 has been reported to be overexpressed in various cancers including GC but also colon, breast and lung cancers, and has been proposed as a potential serum biomarker of gastric cancer. Functionally, OLFM4 has been shown to interact with GRIM19 (a cell-death regulatory protein), cadherins and lectins, and OLFM4 has been shown to inhibit apoptosis and promote tumor growth and invasion. The biological relevance of OLFM4 as a miR-486 target was supported by demonstrating that OLFM4 silencing can reduce gastric cancer cellular proliferation, and that OLMF4 overexpression can rescue the anti-oncogenic effects of miR-486. Interestingly, despite miR-486 being downregulated in both intestinal and diffuse type gastric cancers, we found that OLFM4 overexpression was largely confined to intestinal-type gastric cancers. It is possible that in diffuse type gastric cancers OLFM4 might be targeted by additional miRNAs and not simply mir-486. Consistent with this notion, preliminary bioinformatic analysis suggests that the OLFM4 gene may be targeted by over 400 different mi-RNAs (data not shown). Thus, other mi-RNAs may also act to target OLFM4 in diffuse type GCs, while in intestinal-type gastric cancers, miR-486 regulation of OLFM4 may exert a predominant role. In this regard, it is intriguing to note that OLFM4 has also been recently reported to be a robust marker of intestinal stem cells.

In conclusion, it was shown that miR-486 may act as a novel tumor suppressor mi-RNA in gastric or other type of cancer, and that its down-regulation in gastric tumors, may be required for gastric cancer development and progression.

### APPLICATIONS

A method according to the present invention may be utilized to provide a new treatment for cancer or to enhance an existing treatment.

The disclosed method utilizes small RNA molecules to modulate or inhibit the expression of a particular oligonucleotide encoding for a protein which may be an anti-apoptotic factor and promotes proliferation of abnormal cancer cells.

New treatment based on the disclosed method may have advantages over existing cancer therapies in that the treatment may specifically target cancer cells, and therefore be more effective with reduced side effects as compared to a conventional cancer treatment.

The disclosed method may also be used to enhance an existing cancer treatment, where the cancer cells have shown resistance to chemotherapy drugs, for example.

A silencing oligonucleotide of the present invention comprising a binding motif capable of targeting an oligonucleotide encoding for a protein that functions in cancer may be used to manufacture a medicament that can be administered to a mammal in need of cancer treatment. The silencing oligonucleotide may inhibit the expression of an apoptotic protein, or anchorage independent growth, and/or inhibit migration and invasion of cancer cells, as demonstrated in the Examples.

A method according to the present invention may also be used to induce apoptosis in a cell, for treating a range of cell-proliferative disorders including, but not limited to, cancer.

A method according to the present invention may also be utilized in cancer diagnosis or prognosis. This may be achieved by screening for the downregulation of a silencing oligonucleotide, or the upregulation of an apoptotic factor, of the present invention.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method for treating cancer or enhancing a cancer treatment, the method comprising inhibiting the expression of an oligonucleotide encoding for a protein selected from the group consisting of OLFM4, SP5, TOB1, ARID1A, FBN1 and HAT1, by administering to a mammal in need of cancer treatment an effective amount of at least one silencing oligonucleotide comprising a binding motif with a core nucleotide sequence of UCCUGUAC, or at least one silencing oligonucleotide targeting the 3′ untranslated region of the oligonucleotide encoding for a protein selected from the group consisting of OLFM4, SP5, TOB1, ARID1A, FBN1 and HAT 1.

2. The method according to claim 1, wherein the olignucleotide encoding for a protein is selected from the group consisting of OLFM4, SP5, TOB1 and ARID1A.

3. The method according to claim 2, wherein the oligonucleotide encoding for a protein is selected from the group consisting of OLFM4 and SP5.

4. The method according to claim 1, wherein the at least one silencing oligonucleotide targeting the 3′ untranslated region of the oligonucleotide encoding for a protein selected from the group consisting of OLFM4, SP5, TOB1, ARID1A, FBN1 and HAT1 includes a nucleotide sequence being complementary to the nucleotide sequence of the 3′ untranslated region of the oligonucleotide encoding for a protein.

5. The method according to claim 1, wherein the oligonucleotide encoding for a protein has at least one binding site for binding with the binding motif with a core nucleotide sequence of UCCUGUAC of the silencing oligonucleotide comprising the binding motif.

6. The method according to claim 5, wherein the at least one binding site is located in the 3′ untranslated region of the oligonucleotide encoding for a protein.

7. The method according to claim 6, wherein the oligonucleotide encoding for a protein is an oligonucleotide encoding for the OLFM4 protein.

8. The method according to claim 7, wherein a binding site of the oligonucleotide encoding for the OLFM4 protein comprises a nucleotide sequence of SEQ ID No:3.

9. The method according to claim 1, wherein the silencing oligonucleotide is a mi-RNA, si-RNA, sh-RNA or a precursor thereof.

10. The method according to claim 9, wherein the silencing nucleotide is an mi-RNA selected from the group consisting of hsa-miR-623, hsa-miR-134, hsa-miR-181c, hsa-miR-654-5p, hsa-miR-936, hsa-miR-939, kshv-miR-K12-3, hsa-miR-550, hsa-miR-486-5p, hsa-miR-575, hcmv-miR-UL70-3p, hsa-miR-638, hsvl-miR-H1, hsa-miR-139-3p, hsa-miR-202, hsa-miR-378, hsvl-miR-LAT, hsa-miR-596, hsa-miR-188-5p, hsa-miR-28-3p, hsa-miR-30d, hsa-miR-572, hsa-miR-1225-5p, hsa-miR-345, hsa-miR-30a, hsa-miR-671-5p, hivl-miR-H1, hsa-miR-148a, hsa-miR-222, hsa-miR-10b, hsa-miR-564, hsa-miR-193b, hsa-miR-125a-3p, hsa-miR-370, hsa-miR-375, hsa-miR923, hsa-miR-513c, hsa-miR-513a-5p, hsa-miR-494 and hsa-miR-513b, hsa-miR-486-5p, hsa-let-7d*, hsa-miR-328, hsa-miR-32*, hsa-miR-1227, hsa-miR-206, hsa-miR-1229, hsa-miR-595 and hsa-miR-631, or a precursor of these mi-RNAs.

11. The method according to claim 10, wherein the silencing nucleotide is selected from the group consisting of hsa-miR-375, hsa-miR-148a, miR-671-5p, hsa-miR-30a, hsa-miR-1225-5p, hsa-miR-572, hsa-miR-188-5p, miR-139-3p, hsa-miR-638, hsa-miR-486-5p, hsa-miR-550, miR-939, hsa-miR-936, hsa-miR-181c, miR-134, hsa-miR-623, hsa-let-7d*, hsa-miR-328, hsa-miR-32*, hsa-miR-1227, hsa-miR-206, hsa-miR-1229, hsa-miR-595 and hsa-miR-631, or a precursor of these mi-RNAs

12. The method according to claim 10, wherein the mi-RNA is hsa-miR-486-5p (miR-486) comprising a nucleotide sequence of SEQ ID NO:1.

13. The method according to claim 11, wherein the precursor is a precursor of mi-486 comprising a nucleotide sequence of SEQ ID NO:2.

14. The method according to claim 1, wherein the cancer is selected from the group consisting of gastric cancer, colon cancer, breast cancer and lung cancer.

15. The method according to claim 14, wherein the cancer is gastric cancer.

16. The method according to claim 1, wherein the silencing oligonucleotide comprises a chemical modification of one or more nucleotides.

17. The method according to claim 16, wherein the modification comprises a phosphate backbone modification, a modified sugar moiety, a modified nucleotide or a modified terminal.

18. The method according to claim 17, wherein the phosphate backbone modification is selected from the group consisting of phosphorothioate modification, methylphosphonate modification, phosphotriester modification, phosphordithionate modification and phosphoselenate modification.

19. The method according to claim 1, wherein the silencing oligonucleotide is formulated with a delivery vehicle.

20. The method according to claim 19, wherein the delivery vehicle is a nanoparticle, in the form of a liposome; or a peptide; or an aptamer; or an antibody; or a polyconjugate; or microencapsulation.

21. The method according to claim 20, wherein the lipsome is a stable nucleic acid-lipid particle (SNALP), or dioleoyl phosphatidylcholine (DOPC)-based delivery system, or a lipoplex.

22. The method according to claim 1, wherein the silencing oligonucleotide is formulated for systemic administration.

23. The method according to claim 1, wherein the silencing oligonucleotide is encoded with an expression vector for expression in a mammalian cell.

24. The method according to claim 23, wherein the expression vector is a viral vector selected from the group consisting of a retroviral, adenoviral, lentiviral and adeno-associated viral vector.

25. The method according to claim 23, wherein the mammalian cell is a tumour cell.

26. A method of inducing apoptosis in a mammal in need thereof, the method comprising administering to the mammal an effective amount of at least one silencing oligonucleotide comprising a binding motif of with a core nucleotide sequence of UCCUGUAC to bind and thereby inhibit an anti-apoptotic protein in the mammal.

27. The method according to claim 26, wherein the anti-apoptotic protein is OLFM4.

28. The method according to claim 26, wherein the mammal in need thereof is a mammal with a cell proliferative disorder.

29. The method according to claim 28, wherein the cell proliferative disorder is cancer.

30. The method according to claim 28, wherein the cancer is selected from the group consisting of gastric cancer, colon cancer, breast cancer and lung cancer.

31. A method of screening for cancer, the method comprising screening for a silencing oligonucleotide comprising a binding motif with a core nucleotide sequence of UCCUGUAC or an oligonucleotide encoding for a protein including a matching nucleotide sequence being complementary to the nucleotide sequence of UCCUGUAC.

32. The method of screening for cancer according to claim 31, wherein the method comprises mi-RNA profiling or deep sequencing.

33. The method of claim 31, wherein the cancer is gastric cancer.


SEQUENCE LISTING (4 sequences)

SEQ ID NO: 1 - RNA, 22 nt, Artificial Sequence
  A synthetic hsa-miR-486 (miR-486) (5′ - 3′)
uccuguacugagcugccccgag

SEQ ID NO: 2 - RNA, 68 nt, Artificial Sequence
  A synthetic precursor of miR-486 (5′-3′)
gcauccuguacugagcugccccgaggcccuucaugcugcccagcucggggcagcucaguacaggauac

SEQ ID NO: 3 - RNA, 26 nt, Artificial Sequence
  A synthetic OLFM 4 UTR (5′ - 3′)
gucuagggauucuuuguacaggaaau

SEQ ID NO: 4 - RNA, 23 nt, Artificial Sequence
  A synthetic miR-486 (3′)
gagccccgucgaggucauguccu